(19) United States
(12) Patent Application Publication
(10) Pub. No.: US 2008/0026431 A1
(43) Pub. Date: Jan. 31, 2008

(54) METHOD FOR SACCHARIFICATION OF WOODY BIOMASS

(75) Inventors: Yuji Saito, Tokyo (JP); Norifumi Yamamoto, Tokyo (JP); Hironori Taki, Tokyo (JP); Kazuhide Terashima, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: TAISEI CORPORATION, Shinjuku-ku (JP)

(21) Appl. No.: 11/778,990

(22) Filed: Jul. 17, 2007

(30) Foreign Application Priority Data
Jul. 19, 2006 (JP) 2006-196757

(51) Int. Cl. C12P 19/02; C12P 7/10

(52) U.S. Cl. 435/105; 435/165

(57) ABSTRACT

Provided is an inexpensive and efficient saccharification method for woody biomass and a method of producing ethanol from woody biomass. A method for saccharification of woody biomass comprising hydrolysis treatment and an enzymatic treatment, in which a pretreatment process is performed prior to the enzymatic treatment and said pretreatment includes the steps of: hydrolyzing woody biomass, separating the resultant reaction product into a primary sugar solution and a residue, mixing the residue with an aqueous alkaline solution, and mixing with an oxidant capable of generating active oxygen.

### BACKGROUND OF THE INVENTION

1. Field of the Invention

The present invention relates to a saccharification method for inexpensively and efficiently producing sugars from cellulose or hemicellulose in woody biomass used as a raw material and a method of producing ethanol from the sugars derived form cellulose or hemicellulose.

2. Background Art

In an attempt to overcome global warming, novel energy independent of fossil resources has been progressively developed. Ethanol production from biomass is one of the attempts. In the United States and Brazil, ethanol has already been produced from major agricultural products such as sugarcane and corn, and practically used as eco-fuel for automobile. Also in Japan, researchers have actively studied on use of biomass and developed technology for producing ethanol form biomass. However, unlike in the United State and Brazil, domestic agricultural products and biomass resources suitable for ethanol production are limited in Japan. Therefore, in woodland Japan, it has been desired to develop a technique for producing ethanol from woody biomass including timber.

A process for producing ethanol from biomass roughly consists of two steps. First one is a saccharification step for producing monosaccharides from biomass. The second one is an ethanol production step for producing ethanol from the monosaccharides via fermentation (see JP Patent Publication (Kokai) Nos. 2006-149343A, 2006-87350A, 2005-117942A, and 2004-337099A). If saccharification of hemicellulose and cellulose contained in woody biomass does not proceed in high yield, the overall production of ethanol via fermentation may be significantly affected. For the reason, it has been desired to develop a technique for efficiently obtaining sugars from woody biomass.

Examples of a typical method for saccharification of woody biomass include a method using concentrated sulfuric acid (concentrated sulfuric acid method) and a method using dilute sulfuric acid (dilute sulfuric acid method) may be mentioned. The concentrated sulfuric acid method is high in saccharification efficiency but requires high equipment cost because sulfuric acid whose concentration is as high as 70 to 80% is used at a temperature of about 50° C. to 100° C. In addition, its operational safety is a matter of concern. On the other hand, the dilute sulfuric acid method does not require high equipment cost but is low in saccharification efficiency.

Now, a saccharification method according to the dilute sulfuric acid method will be explained including specific problems by way of typical woody biomass, timber.

Timber (hereinafter referred to as “wood”) is formed of three major components, namely, hemicellulose, cellulose and lignin. In the first stage for obtaining a monosaccharide, wood is mixed with dilute sulfuric acid and the mixture is subjected to hydrolytic decomposition performed at a temperature of 140 to 220° C. for 3 to 20 minutes. In this manner, hemicellulose contained in wood is hydrolyzed to obtain monosaccharides derived from hemicellulose. Examples of the monosaccharides derived from hemicellulose include pentoses such as xylose and arabinose and hexoses such as glucose and galactose and mannose.

After saccharification of hemicellulose, a residue containing lignin and cellulose is obtained as a solid component. In the second stage, the residue obtained in the first stage is hydrolyzed with dilute sulfuric acid under severer temperature/pressure conditions than those of the first stage. In this manner, a hexose namely glucose is obtained from cellulose.

The saccharification yield from hemicellulose in the first stage of the dilute sulfuric acid method is 90 to 95%, whereas the saccharification yield of cellulose in the second stage is at most about 30 to 35%. This is because glucose obtained by saccharification is excessively decomposed to produce formic acid, levulinic acid and hydroxymethylfurfural (HMF). The presence of the excessively decomposed products affects fermentation performed later.

To overcome the technical problems involved in saccharification with dilute sulfuric acid or concentrated sulfuric acid, an attempt has been aggressively made to obtain sugar enzymatically from woody biomass. However, when cellulose-hydrolyzing enzyme (cellulase) is directly injected to woody biomass, the enzyme is adsorbed tightly to a component of woody biomass, lignin. As a result, a large amount of an enzyme is required for saccharification of target cellulose. It is unfavorable from an economic point of view.

On the other hand, there are reports that not woody biomass but biomass such as agricultural products and aquatic plants, is previously treated with alkali and hydrogen peroxide, and then subjected to saccharification with cellulase (J. Michael Gould, vol. XXVI, p. 046-052, 1984; D. Mishima et al., Bioresource Technology, vol. 97, p. 2166-2172, 2006). According to these documents, saccharification of wheat straw, cone cobs, cone stalks, cone husks, and water hyacinth, etc. proceeds in very high yield, whereas, that of kenaf and oak wood proceeds in low yield. However, even if wood is pretreated directly with alkali and hydrogen peroxide and then treated with cellulase, high saccharification yield cannot be obtained (see Example 1 (described later), No. 5).

An object of the invention is to provide an inexpensive and efficient method for saccharification of woody biomass and a method of producing ethanol from the woody biomass.

### SUMMARY OF THE INVENTION

The present inventors have conducted intensive studies to solve the aforementioned problems. They hydrolyzed woody biomass and separated into a liquid component (primary sugar solution) and a solid component (residue). The residue was treated with alkali and hydrogen peroxide and thereafter treated with a general amount of cellulase used in the art to obtain a secondary sugar solution. As a result, they found that saccharification yield of the secondary sugar solution is high. Based on the finding, they accomplished the invention.

More specifically, the present invention relates to the following items (1) to (8).

(1) A method for saccharification of woody biomass comprising hydrolysis treatment and an enzymatic treatment, in which a pretreatment process is performed prior to the enzymatic treatment and said pretreatment includes the steps of: hydrolyzing woody biomass, separating the resultant reaction product into a primary sugar solution and a residue, mixing the residue with an aqueous alkaline solution, and mixing with an oxidant capable of generating active oxygen.

(2) A method for saccharification of woody biomass comprising hydrolysis treatment and an enzymatic treatment, in which a pretreatment process is performed prior to the enzymatic treatment and said pretreatment includes the steps of: hydrolyzing woody biomass, separating the resultant reaction product into a primary sugar solution and a residue, mixing the residue with an aqueous alkaline solution to obtain a mixture, separating the mixture into a secondary residue and a solution, and mixing an oxidant capable of generating active oxygen with the obtained secondary residue in maintaining alkaline state.

(3) A method for saccharification of woody biomass comprising hydrolysis treatment and an enzymatic treatment, in which a pretreatment process is performed prior to the enzymatic treatment and said pretreatment includes the steps of: hydrolyzing woody biomass, separating the resultant reaction product into a primary sugar solution and a residue, mixing the residue with an aqueous alkaline solution, and mixing with an oxidant capable of generating active oxygen; and subjecting a solid component of the resultant mixture is subjected to an enzymatic treatment with cellulase.

(4) A method for saccharification of woody biomass comprising hydrolysis treatment and an enzymatic treatment, in which a pretreatment process is performed prior to the enzymatic treatment and said pretreatment includes the steps of: hydrolyzing woody biomass, separating the resultant reaction product into a primary sugar solution and a residue, mixing the residue with an aqueous alkaline solution to obtain a mixture, separating the mixture into a secondary residue and a solution, and mixing an oxidant capable of generating active oxygen with the obtained secondary residue in maintaining alkaline state; and subjecting a solid component of the resultant mixture is subjected to an enzymatic treatment with cellulase.

In the methods of items (1) to (4), hydrolysis may be performed with dilute sulfuric acid.

Furthermore, in the methods of items (1) to (4), the residue may be mixed with an aqueous alkaline solution at pH of e.g., 10 to 13, at a temperature of e.g., normal temperature. Examples of the aqueous alkaline solution to be used herein include, but not limited to, an aqueous solution of sodium hydroxide.

In the methods of items (1) to (4), examples of the oxidant generating active oxygen that can be used include hydrogen peroxide, and more specifically, hydrogen peroxide of 0.1% or more.

Moreover, in the methods of items (1) to (4), the solid component is subjected to an enzymatic treatment with cellulase to obtain a secondary sugar solution. Alternatively, the solid component is subjected to an enzymatic treatment with cellulase and an ethanol fermentation treatment to produce ethanol.

(5) A method of producing ethanol from woody biomass comprising performing a pretreatment process, which includes the steps of: hydrolyzing woody biomass, separating the resultant reaction product into a primary sugar solution and a residue, mixing the residue with an aqueous alkaline solution, and mixing with an oxidant capable of generating active oxygen; subjecting a solid component of the mixture obtained to enzymatic treatment with cellulase to obtain a secondary sugar solution; and subjecting the primary sugar solution and/or the secondary sugar solution to ethanol fermentation.

(6) A method of producing ethanol from woody biomass comprising performing a pretreatment process, which includes the steps of: hydrolyzing woody biomass, separating the resultant reaction product into a primary sugar solution and a residue, mixing the residue with an aqueous alkaline solution to obtain a mixture, separating the mixture into a secondary residue and a solution, and mixing an oxidant capable of generating active oxygen with the obtained secondary residue in maintaining alkaline state; subjecting a solid component of the mixture obtained to enzymatic treatment with cellulase to obtain a secondary sugar solution; and subjecting the primary sugar solution and/or the secondary sugar solution to ethanol fermentation.

(7) A method of producing ethanol from woody biomass comprising performing a pretreatment process, which includes the steps of: hydrolyzing woody biomass, separating the resultant reaction product into a primary sugar solution and a residue, mixing the residue with an aqueous alkaline solution, and mixing with an oxidant capable of generating active oxygen; and subjecting a solid component of the mixture obtained to an enzymatic treatment with cellulase and ethanol fermentation treatment.

(8) A method of producing ethanol from woody biomass comprising performing a pretreatment process, which includes the steps of: hydrolyzing woody biomass, separating the resultant reaction product into a primary sugar solution and a residue, mixing the residue with an aqueous alkaline solution to obtain a mixture, separating the mixture into a secondary residue and a solution, and mixing an oxidant capable of generating active oxygen with the obtained secondary residue in maintaining alkaline state; and subjecting a solid component of the mixture obtained to an enzymatic treatment with cellulase and ethanol fermentation treatment.

In the method according to items (7) and (8), the enzymatic treatment with cellulase and an ethanol fermentation treatment are simultaneously performed by using a cellulase-producing microorganism and ethanol-fermenting microorganism and using the solid component as a common raw material.

The present invention makes it possible to provide an inexpensive and efficient method for saccharification of woody biomass. According to the saccharification method of the invention, the saccharification yields of hemicellulose and cellulose contained in woody biomass can be greatly improved and energy can be saved because the reaction of saccharification is performed under normal temperature/pressure conditions. Furthermore, in each step of the saccharification method of the invention, since the concentration of an alkali compound or an oxidant is low, the equipment can be simplified and its operational safety is high. Moreover, the present invention provides a method of producing ethanol. In the ethanol production method of the present invention, woody biomass can be efficiently converted into energy. Therefore, it is effective to recycle a natural resource.

### BRIEF DESCRIPTION OF THE DRAWINGS

FIG. 1 shows a basic flow of a saccharification method according to the present invention and a pretreatment process;

FIG. 2 shows an application example of the saccharification method;

FIG. 3 shows an application example of the saccharification method and the ethanol production method;

FIG. 4 shows other application examples of the saccharification method and the ethanol production method;

FIG. 5 is a graph showing the relationship between pH of an aqueous alkaline solution and saccharification yield;

FIG. 6 is a graph showing the relationship between hydrogen peroxide concentration and saccharification yield; and

FIG. 7 is a graph showing the relationship between additive amount of hydrogen peroxide concentration and saccharification yield.

### DETAILED DESCRIPTION OF THE PREFERRED EMBODIMENTS

The present invention will be more specifically described, below.

In a method for saccharification of woody biomass according to the present invention (hereinafter sometimes referred to as “the present saccharification method”), woody biomass is hydrolyzed and thereafter a primary sugar solution and a residue are obtained. The residue is subjected to a pretreatment (AO treatment) performed with an aqueous alkaline solution and an oxidant capable of generating active oxygen. By virtue of the pretreatment, sugar can be highly efficiently produced in the enzymatic treatment step (performed later). The present invention also provides a method of producing ethanol from the sugar obtained from woody biomass as mentioned above.

1. Saccharification method

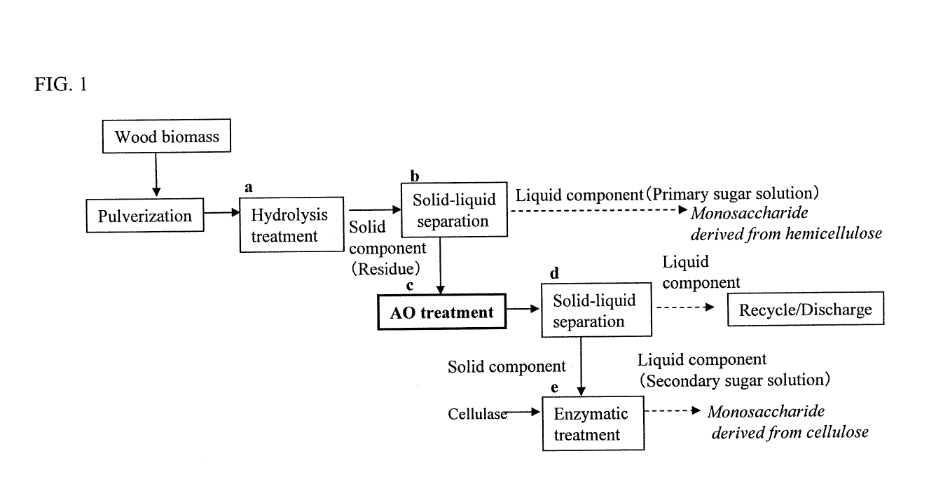

FIG. 1 shows a basic flow of a pretreatment method and a saccharification method according to the present invention. More specifically, the present saccharification method includes the following steps:

(a) step of hydrolyzing woody biomass (hydrolysis step),

(b) step of separating the resultant reaction product into a primary sugar solution and a residue (solid-liquid separation step),

(c) step of treating the residue using an aqueous alkaline solution and an oxidant capable of generating active oxygen (AO treatment step),

(d) step of separating the mixture into a liquid component and a solid component (solid-liquid separation step), and

(e) step of enzymatically treating the solid component with cellulase (enzymatic treatment step).

The present saccharification method is characterized in the pretreatment step (c).

The term “saccharification” used in the invention refers to decomposing hemicellulose and cellulose contained in woody biomass to monosaccharides or disaccharides. Hemicellulose contained in woody biomass is decomposed by the hydrolysis step (a). Sugar derived from the hemicellulose contained in a primary sugar solution can be obtained in the step (b). Cellulose contained in woody biomass is decomposed by the treatments of steps (c) to (e) and sugar derived from cellulose contained in a secondary sugar solution can be obtained.

(a) Hydrolysis step

In the present saccharification method, first, woody biomass is mixed with an appropriate acid or alkali. The mixture is allowed to react by raising the temperature of the mixture to a predetermined temperature. In this manner, hemicellulose contained in the woody biomass is hydrolyzed.

Examples of the woody biomass to be used as a raw material in the present invention include, but not particularly limited to, building material waste, packaging material waste, felled trees, sawdust, thinning tree, wood chip, rice straw, bark, slash, unused trees, and wastewood. Woody biomass may consist of a single type or a plurality of types of wood resources. Furthermore, woody biomass may include a small amount of impurities other than a wood resource.

Woody biomass is preferably pulverized before hydrolysis treatment. Woody biomass can be pulverized, for example, by a refiner or a wood chopper.

The hydrolysis reaction may be performed in appropriate conditions in accordance with a known method in the art, either with acid or alkali. For example, when hydrolysis is performed with acid, sulfuric acid, preferably, dilute sulfuric acid, may be used. Hydrolysis is performed, with 0.1 to 5%, preferably 0.5 to 3% dilute sulfuric acid at a temperature of about 140 to 220° C., preferably 160 to 210° C. for 1 to 20 minutes, preferably about 5 to 10 minutes.

(b) Solid-liquid separation step

Subsequently, the reaction product obtained in the hydrolysis step is subjected to a solid-liquid separation to obtain a primary sugar solution and a residue. The solid-liquid separation is performed by a known method in the art, such as filter press, vibrating screen, centrifugation, or membrane separation. The yield of a monosaccharide obtained from the primary sugar solution is about 95% based on the hemicellulose contained in woody biomass.

(c) AO treatment step

Subsequently, the residue is subjected to AO treatment. More specifically, the residue is mixed with an aqueous alkaline solution and then with an oxidant capable of generating active oxygen.

As the aqueous alkaline solution, any type of aqueous alkaline solution may be used, including, aqueous solutions of sodium hydroxide, potassium hydroxide, calcium hydroxide and calcium oxide. The aqueous alkaline solution to be used herein may have a pH value within the range of 9.5 to 13.5, preferably 10 to 13, more preferably, 11 to 12.5. The aqueous alkaline solution is mixed in an amount of about 100 to 2000 mL, preferably 200 to 1000 mL, more preferably 300 to 500 mL relative to 100 g of the residue obtained after hydrolysis treatment and continuously stirred preferably at normal temperature (about 20 to 30° C.) for about 1 to 30 hours, preferably 1 to 15 hours while controlling the pH of the mixture at 10 to 13.

As a next step, an oxidant capable of generating active oxygen is added to the aqueous alkaline solution mixture and mixed well. As the oxidant capable of generating active oxygen, any oxidant known in the art may be used. Specific examples thereof include hydrogen peroxide, persulfate, percarbonate, peracetate, ozone, and sodium peroxide. The oxidant capable of generating active oxygen may be added in an appropriate amount and stirred, for example, for 1 to 30 hours. More specifically, when hydrogen peroxide is used, hydrogen peroxide is added so as to obtain a final concentration of the mixture of 0.1% or more and stirred for about 1 to 30 hours and preferably 1 to 10 hours.

AO treatment to the residue is not limited to the above described procedure. The AO treatment may be a treatment comprising steps of mixing the residue with an aqueous alkaline solution to obtain a mixture, separating the mixture into a secondary residue and a solution, and mixing an oxidant capable of generating active oxygen with the obtained secondary residue in maintaining alkaline state. Specifically, the secondary residue can be obtained by a method that comprises steps of mixing the residue and aqueous alkaline solution, static standing the resultant mixture, and removing a liquid phase thereof, or by a method that comprises steps of mixing the residue and aqueous alkaline solution, precipitating a solid component by a centrifugation, and removing a liquid phase thereof. In addition, in the case of this procedure, the secondary residue is maintained in alkaline state in the step of mixing the secondary residue and the oxidant. Specifically, the oxidant such as hydrogen peroxide may be mixed with the secondary residue together with alkaline chemicals such as sodium hydroxide.

By the AO treatment, lignin of the solid component can be decomposed into low-molecular weight compounds or eliminated. As a result, the efficiency of the enzymatic treatment with cellulase (described later) can be greatly improved. Especially, by the AO treatment that comprises steps of mixing an oxidant capable of generating active oxygen with the obtained secondary residue in maintaining alkaline state, lignin of the solid component can be decomposed into low-molecular weight compounds or eliminated using comparatively small amount of the oxidant.

(d) Solid-liquid separation step

Subsequently, the mixture obtained after the pretreatment (AO pretreatment) is separated into a liquid component and a solid component. The solid-liquid separation is performed by a known method in the art in the same manner as above. The solid component obtained after the AO treatment is subjected to the enzymatic treatment step. The liquid component may be discharged or fed back to the AO treatment for recycle. When the liquid component (obtained by solid-liquid separation after the AO treatment) is reused, the pH of the liquid component, which has been lowered after addition of an oxidant, is adjusted with alkali so as to fall within the range of 10 to 13. Thereafter, another residue freshly obtained after hydrolysis is mixed in the aforementioned mixing ratio and stirred for a predetermined time. Thereafter, an oxidant capable of generating active oxygen is added and mixed. In this manner, lignin contained in the solid component can be decomposed into lower molecular-weight compounds or eliminated.

(e) Enzymatic treatment step

In this step, the solid component thus obtained is enzymatically treated with cellulase to hydrolyze cellulose of the solid component to monosaccharides. Cellulase to be used herein is not particularly limited as long as it can efficiently hydrolyze cellulose to a hexose. More specifically, cellulase may be derived from a plant or an animal, chemically modified and produced by a genetic recombination technique. Note that the temperature and time period of the reaction with cellulase and the amount of cellulase to be used in the reaction may vary depending upon the type of cellulase. However, one skilled in the art can appropriately choose them depending upon the type of cellulase.

Alternatively, the solid component may be used as a raw material for fermentation by a cellulase-producing microorganism to hydrolyze cellulose of the solid component into monosaccharides. In this manner, a secondary sugar solution can be obtained. Such a cellulase-producing microorganism is known in the art. Examples of the cellulase-producing microorganism include Aspergillus niger, A. foetidus, Alternaria alternata, Chaetomium thermophile, C. globosus, Fusarium solani, Irpex lacteus, Neurospora crassa, Cellulomonas fimi, C. uda, Erwinia chrysanthemi, Pseudomonas fluorescence and Streptmyces flavogriseus, which are described, for example in “cellulose resource-technological development for advanced use and fundamental thereof” edited by Tetsuo Koshijima, published by Japan Scientific Societies Press, 1991.

The amount of monosaccharides contained in the secondary sugar solution obtained in the abovementioned treatment reaches 80 to 90% based on the cellulose contained in the solid component.

The solid component (obtained after the AO treatment) may be enzymatically treated with cellulase as mentioned above. Besides this, using the solid matter as a raw material, cellulase production by a cellulase-producing microorganism and ethanol fermentation by an ethanol-fermenting microorganism may be simultaneously performed to produce ethanol.

By the dilute sulfuric acid treatment (first saccharification stage), the AO treatment (second saccharification stage) and enzymatic treatment, monosaccharides can be obtained in high yield from hemicellulose and cellulose contained in woody biomass. A microorganism capable of producing ethanol uses the monosaccharides thus produced as a raw material for fermentation.

2. Production of ethanol

Ethanol fermentation is performed utilizing the sugar (primary sugar solution and secondary sugar solution) obtained in the saccharification method mentioned above as a raw material to obtain ethanol.

The primary sugar solution contains sugar derived from hemicellulose including pentoses such as xylose and arabinose and hexoses such as glucose, galactose and mannose. A hexose can be easily converted into ethanol by yeast or the like. A pentose can be converted into ethanol in accordance with an ethanol production method known in the art.

The secondary sugar solution contains sugar derived from cellulose, including a hexose such as glucose and can be easily converted into ethanol by yeast or the like.

The ethanol fermentation of the hexose may be performed in accordance with an ethanol production method known in the art by yeast or bacteria to which a gene required for ethanol production is inserted by genetic recombination.

The ethanol fermentation is performed by genetically modified Escherichia coli, which can utilize both pentose and hexose but does not produce ethanol, to which a microbial gene involved in ethanol production is introduced by genetic recombination or performed by genetically modified ethanol-fermenting bacteria belonging to the genus Zymomonas, to which a gene involved in pentose metabolism is introduced by genetic recombination (see, for example, National Publication of International Patent Application Nos. 5-502366 and 6-504436). Alternatively, a pentose and a hexose may be fermented to obtain ethanol and carbon dioxide and ethanol is recovered (JP Patent Publication (Kokai) No. 2006-111593A).

One may appropriately set conditions for ethanol fermentation skilled in the art depending upon the type of sugar used as a raw material and the type of ethanol-fermenting microorganism. The ethanol fermentation from the primary sugar solution and the secondary sugar solution may be performed separately or in a mixture thereof.

3. Application Example

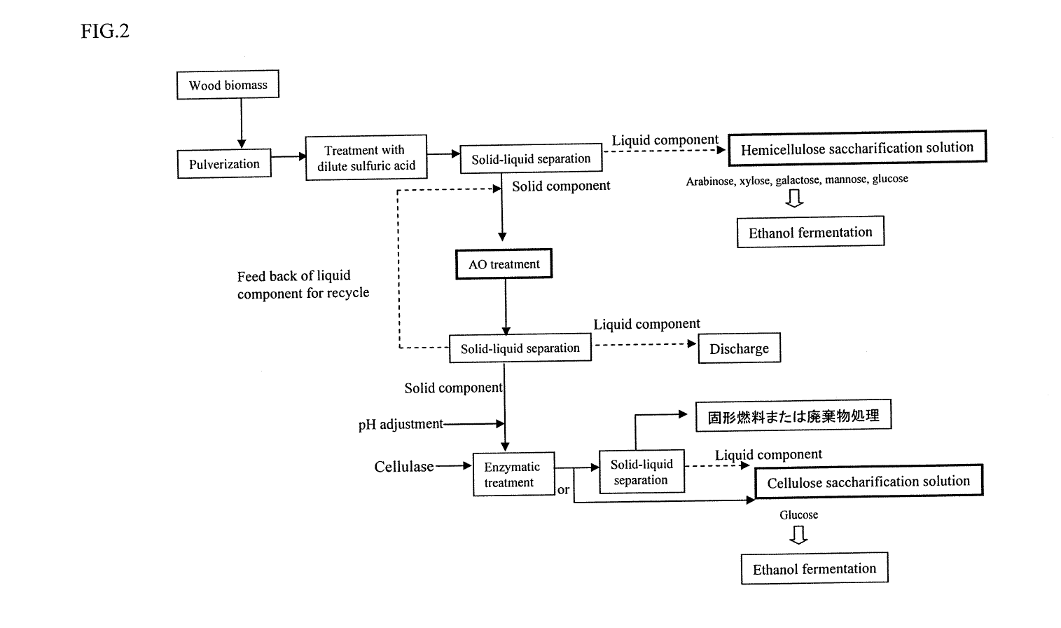

Preferred embodiments of a method for saccharification of woody biomass and an ethanol production method to which the present invention is applied are shown in FIGS. 2, 3 and 4.

(1) Application Example 1 (see FIG. 2)

Woody biomass is pulverized and hydrolyzed with dilute sulfuric acid at 140 to 220° C. for 3 to 20 minutes (expressed as “dilute sulfuric acid treatment” in FIG. 2). Solid-liquid separation is performed. The resultant liquid component is a solution containing monosaccharides derived from hemicellulose (hemicellulose saccharification solution), including pentoses such as xylose and the like. The saccharification solution containing pentoses is used as a raw material of fermentation by an ethanol-fermenting microorganism. The solid component obtained by the solid-liquid separation is subjected to the AO treatment performed under normal temperature/normal pressure conditions. The resultant reaction product is subjected to solid-liquid separation. The liquid component obtained herein is fed back to the AO treatment for recycle. To the solid component, cellulase is added after its pH is adjusted within the optimal working range for cellulase (expressed as “enzymatic treatment” in FIG. 2). In this way, cellulose contained in the solid component is enzymatically decomposed to monosaccharides. If necessary, solid-liquid separation is performed to obtain a saccharification solution. The saccharification solution is used as a raw material for fermentation by an ethanol-fermenting microorganism. Note that when the solid liquid separation is performed, the solid component separated is used as a fuel or discarded.

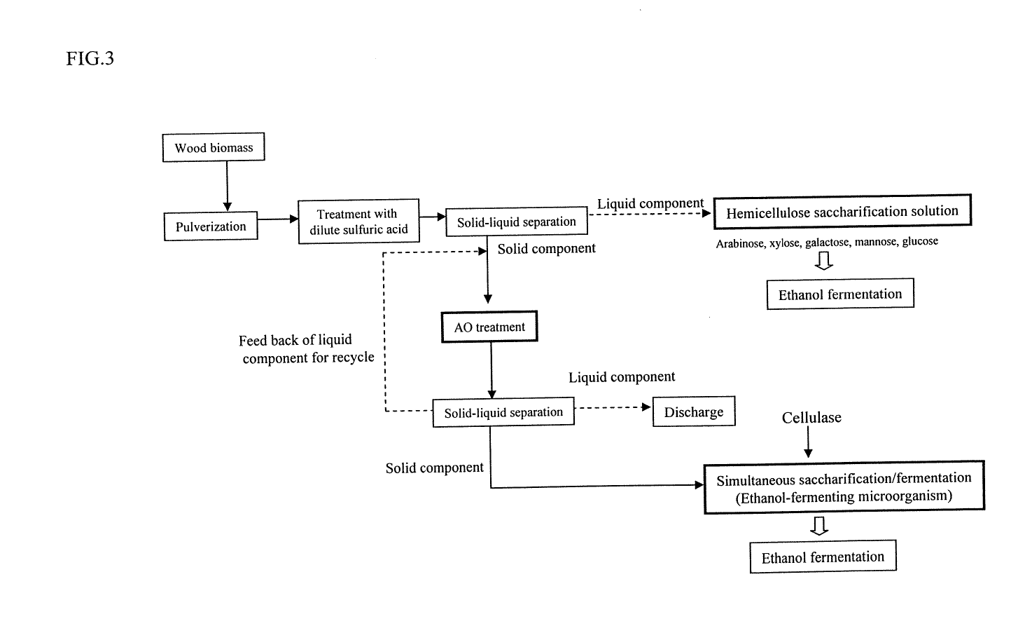

(2) Application Example 2 (see FIG. 3)

Woody biomass is pulverized and hydrolyzed with dilute sulfuric acid at 140 to 220° C. for 3 to 20 minutes (expressed as “dilute sulfuric acid treatment” in FIG. 3). Solid-liquid separation is performed. The resultant liquid component is a solution containing monosaccharides derived from hemicellulose (hemicellulose saccharification solution), including pentoses such as xylose and the like. The saccharification solution containing pentoses is used as a raw material of fermentation by an ethanol-fermenting microorganism. The solid component obtained by the solid-liquid separation is subjected to the AO treatment performed under normal temperature/normal pressure conditions. The resultant reaction product is subjected to solid-liquid separation. The liquid component obtained herein is fed back to the AO treatment for recycle. The obtained solid component is mixed with a culture solution of an ethanol-fermenting microorganism containing a predetermined amount of cellulase and used as a raw material for simultaneous saccharification/fermentation.

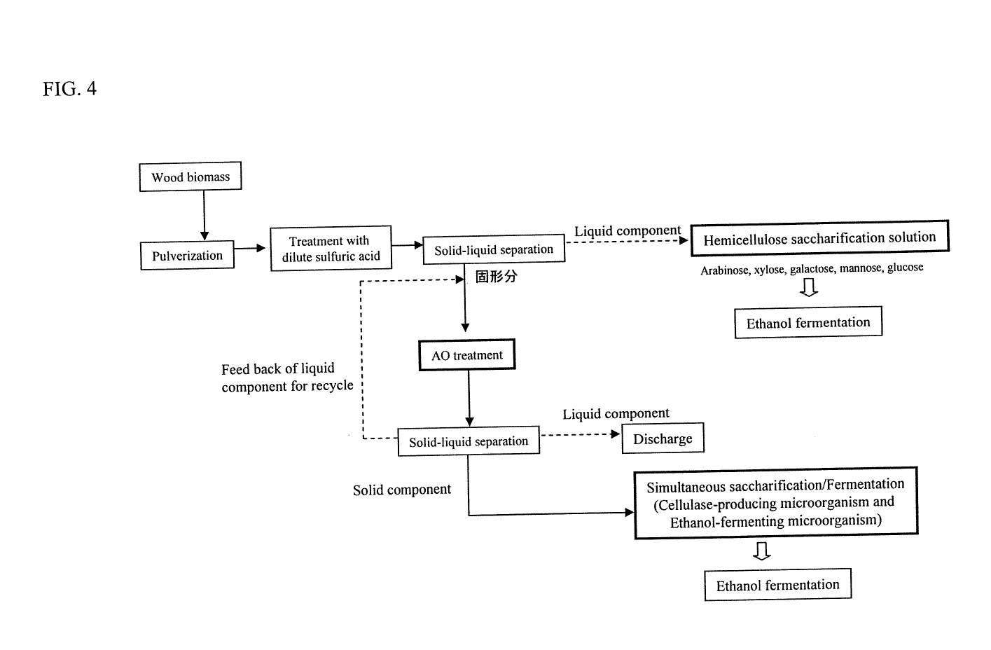

(3) Application Example 3 (see FIG. 4)

Woody biomass is pulverized and hydrolyzed with dilute sulfuric acid at 140 to 220° C. for 3 to 20 minutes (expressed as “dilute sulfuric acid treatment” in FIG. 4). Solid-liquid separation is performed. The resultant liquid component is a solution containing monosaccharides derived from hemicellulose (hemicellulose saccharification solution), including pentoses such as xylose and the like. The saccharification solution containing pentoses is used as a raw material of fermentation by an ethanol-fermenting microorganism. The solid component obtained by the solid-liquid separation is subjected to the AO treatment performed under normal temperature/normal pressure conditions. The resultant reaction product is subjected to solid-liquid separation. The liquid component obtained herein is fed back to the AO treatment for recycle. The obtained solid component is used as a raw material for simultaneous saccharification/fermentation by a cellulase-producing microorganism and an ethanol-fermenting microorganism.

The present invention will be more specifically described by ways of Examples; however, the technical scope of the present invention is not limited to the Examples.

### EXAMPLE 1

In this Example, saccharification treatment was performed by use of pulverized building-material waste as woody biomass. The composition of the pulverized building material waste was previously analyzed. As a result, the pulverized building material waste was composed of 27% of hemicellulose, 40% of cellulose and 33% of lignin+ash. The pulverized wood sample was treated under each of the conditions shown in Table 1 and saccharification efficiency under each condition was obtained. The conditions shown in No. 4 of Table 1 correspond to those of the present invention.

The pulverized wood sample (No. 1) was mixed with 2% dilute sulfuric acid and the mixture was allowed to react at 170° C. for 10 minutes (dilute sulfuric acid treatment) in the first saccharification stage. Subsequently, the resultant residue was pulverized again by a refiner and mixed with 2% dilute sulfuric acid at 220° C. for 10 minutes (dilute sulfuric acid treatment).

The sample (No. 2) was mixed with 2% dilute sulfuric acid (dilute sulfuric acid treatment) in the first saccharification stage in the same manner as in sample No. 1. To the residue pulverized again by a refiner, cellulase (GC 220 manufactured by Genencor, Inc) was added so as to obtain a concentration of 15 FPU/residue (g) on a dry base. The mixture was stirred at 40° C. for 144 hours (enzymatic treatment). Note that the “FPU” indicates the enzyme activity of cellulase for producing 10.8 mg of glucose in 60 minutes from a filter paper. A saccharification speed: 1 FPU=10.8 mg/h.

The sample (No. 3) was treated with dilute sulfuric acid in the same manner as in sample No. 1 in the first saccharification stage. The resultant residue was pulverized again by a refiner and mixed with an aqueous solution of sodium hydroxide, which was adjusted at pH of 12.5, at normal temperature for 15 hours (alkaline treatment). The mixture solution was subjected to solid-liquid separation. To the resultant residue, cellulase (GC 220 manufactured by Genencor, Inc) was added so as to obtain a concentration of 15 FPU/residue (g) on a dry base. The mixture was stirred at 40° C. for 144 hours (enzymatic treatment).

The sample (No. 4) was treated with dilute sulfuric acid in the same manner as in Sample No. 1 in the first saccharification stage. The resultant residue was pulverized again by a refiner and mixed with an aqueous solution of sodium hydroxide, which was adjusted at pH of 12.5, at normal temperature for 3 hours. To the mixture, hydrogen peroxide was added so as to obtain a final concentration of 1%. The mixture was further stirred for 12 hours (AO treatment) and subjected to solid-liquid separation. To the resultant residue, cellulase (GC 220 manufactured by Genencor, Inc) was added so as to obtain a concentration of 15 FPU/residue (g) on a dry base. The mixture was stirred at 40° C. for 144 hours (enzymatic treatment).

The pulverized wood sample (No. 5) was mixed with an aqueous solution of sodium hydroxide, which was adjusted to pH of 12.5, at normal temperature for 3 hours. Thereafter, hydrogen peroxide was added to the mixture so as to obtain a final concentration of 1%. The mixture was further stirred for 12 hours (AO treatment) and subjected to solid-liquid separation. To the resultant residue, cellulase (GC 220 manufactured by Genencor, Inc) was added so as to obtain a concentration of 15 FPU/residue (g) on a dry base. The mixture was stirred at 40° C. for 144 hours (enzymatic treatment).

The pulverized wood sample (No. 6) was mixed with city water. The mixture was stirred at normal temperature for 15 hours. The mixture was subjected to solid-liquid separation. To the resultant residue, cellulase (GC 220 manufactured by Genencor, Inc) was added so as to obtain a concentration of 15 FPU/residue (g) on a dry base. The mixture was stirred at 40° C. for 144 hours (enzymatic treatment).

TABLE 1
Experimental conditions

| | First saccharification | |
|---|---|---|
| | stage (saccharification | Second saccharification stage |
| | of hemicellulose) | (saccharification of cellulose) |
| No. 1 | Dilute sulfuric acid treatment | Dilute sulfuric acid treatment |
| No. 2 | Dilute sulfuric acid treatment | Enzymatic treatment |
| No. 3 | Dilute sulfuric acid treatment | Alkaline treatment + enzymatic |
| | | treatment |
| No. 4 | Dilute sulfuric acid treatment | AO treatment + enzymatic |
| | | treatment |

| | |
|---|---|
| No. 5 | AO treatment + enzymatic treatment |
| No. 6 | Enzymatic treatment |

The concentrations of monosaccharides of the filtrates in the first and second saccharification stages obtained in the aforementioned treatment conditions were determined (measured) by GPC (gel permeation chromatography). As a result, saccharification yields of hemicellulose and cellulose contained in each of the pulverized wood samples were obtained. The saccharification yields obtained under individual conditions are shown in Table 2.

TABLE 2
Results of saccharification yield

| | Saccharification | Saccharification | | |
|---|---|---|---|---|
| | yield (wt %) | yield (wt %) | Saccharification yield | Saccharification |
| | in first | in second | (wt %) | yield |
| | saccharification | saccharification | (in terms of | (wt %) |
| | stage (in terms of | stage (in terms of | hemicellulose and | (in terms of |
| | hemicellulose) | cellulose) | cellulose) | wood) |
| No. 1 | 95 | 35 | 59 | 40 |
| No. 2 | 95 | 20 | 50 | 34 |
| No. 3 | 95 | 23 | 52 | 35 |
| No. 4 | 95 | 80 | 86 | 58 |
| No. 5 | | | 33 | 22 |
| No. 6 | | | 22 | 15 |

As a result, sample of No. 4 to which the present invention is applied exhibited a saccharification yield of 95% in the first saccharification stage, 80% in the second saccharification stage, and 58% per weight of pulverized wood.

### EXAMPLE 2

In this Example, an optimal pH for the alkaline treatment was obtained using the residue (containing 49% of cellulose and 51% of lignin, etc.) obtained in the first saccharification stage (dilute sulfuric acid treatment) of Example 1. Eleven 200 mL culture bottles were prepared. To each of the bottles, 5 g of the residue obtained after the dilute sulfuric acid treatment was placed. Further, 100 mL of an aqueous alkaline solution, whose pH was adjusted by 10% aqueous solution of sodium hydroxide to 8.5 to 13.5, was added. The mixture was stirred at normal temperature for 3 hours. Thereafter, hydrogen peroxide was poured in each of the culture bottles so as to obtain a final concentration of 1%. The mixture was maintained as it was for 12 hours.

After completion of the reaction, the residue was subjected to solid-liquid separation. The residue was mixed with an acetic acid buffer at pH 4.8 and treated with cellulase. Note that the addition amount of cellulase (GC 220 manufactured by Genencor, Inc) was 15 FPU per residue (g) on a dry base. After addition of cellulase, a reaction was performed while stirring at 40° C. for 144 hours. After completion of the reaction, the amount of glucose was determined by GPC and the saccharification yield from cellulose was obtained.

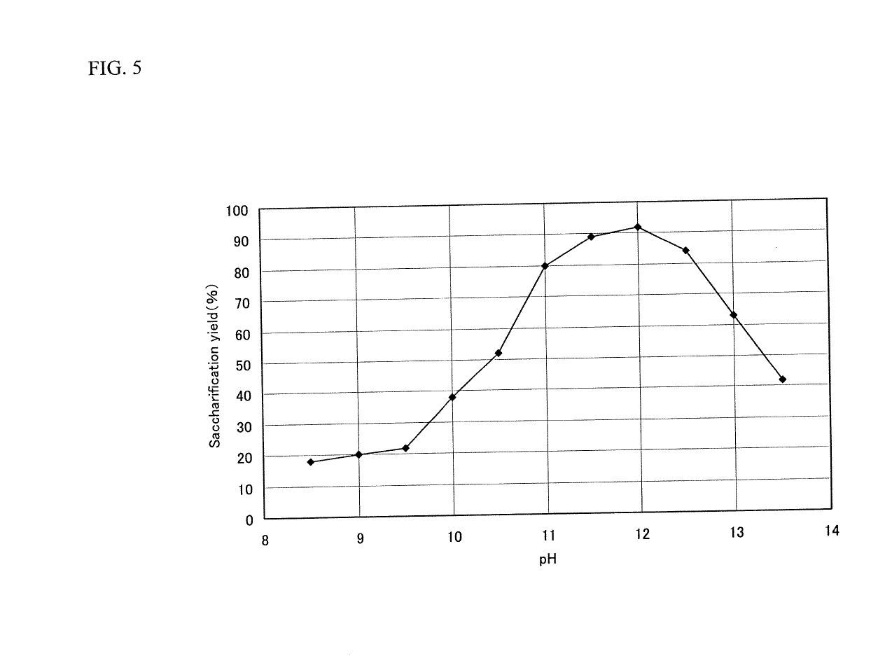

FIG. 5 shows the relationship between pH of an aqueous alkaline solution used in a pretreatment and saccharification yield in terms of cellulose. The saccharification yield was greatly varied depending upon the pH of an aqueous alkaline solution. The saccharification yield was confirmed to be particularly improved at pH of 9.5 or more.

When saccharification of cellulose is performed with a conventional dilute sulfuric acid method, a saccharification yield is 30 to 35%. From this, the present invention makes it possible to produce monosaccharides more efficiently than the dilute sulfuric acid method by use of an aqueous alkaline solution at pH 10 to 13.

### EXAMPLE 3

In this Example, effect of a hydrogen peroxide solution was checked by use of the residue (49% of cellulose and 51% of lignin, etc.) obtained in the first saccharification stage (dilute sulfuric acid treatment) of Example 1. Nine 200 mL culture bottles were prepared. To each of the bottles, 5 g of the residue obtained after the treatment of dilute sulfuric acid was placed. Further, 100 mL of an aqueous alkaline solution, whose pH was adjusted with sodium hydroxide to 12, was added. The mixture was stirred at normal temperature for 3 hours. A 31% hydrogen peroxide solution was added to individual bottles so as to obtain final concentrations of 0%, 0.1%, 0.3%, 0.5%, 0.8%, 1.0%, 2.0%, 5.0%, and 10.0%, respectively. The mixture solutions were maintained as they were for 12 hours.

After completion of the reaction, the residue was subjected to solid-liquid separation. The residue was mixed with an acetic acid buffer at pH 4.8 and treated with cellulase. Note that the addition amount of cellulase (GC 220 manufactured by Genencor, Inc) was 15 FPU per residue (g) on a dry base. After addition of cellulase, a reaction was performed while stirring at 40° C. for 144 hours. After completion of the reaction, the amount of glucose was determined by GPC and the saccharification yield from cellulose was obtained.

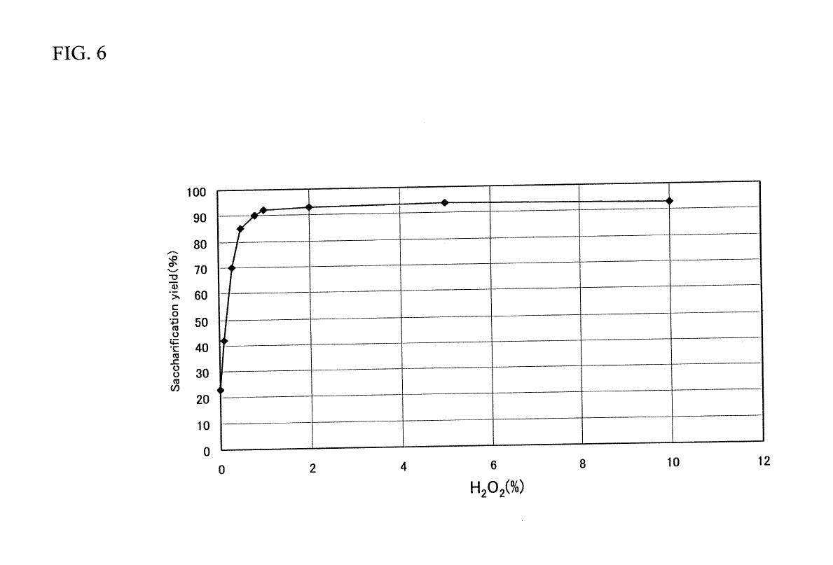

FIG. 6 shows the relationship between the concentrations of hydrogen peroxide used in a pretreatment and saccharification yield (in terms of cellulose). In the reaction where no hydrogen peroxide was added, a saccharification yield of cellulose alone was 23%. However, the saccharification yield was sharply increased by using hydrogen peroxide in a pretreatment. The saccharification yield was almost equal at a hydrogen peroxide concentration of 1% or more.

As described above, the conventional method for saccharification of cellulose with dilute sulfuric acid provides a saccharification yield of 30 to 35%. However, the method of the present invention provides monosaccharides more efficiently than the dilute sulfuric acid method if the hydrogen peroxide concentration is 0.1% or more.

### Example 4

In this Example, treatment efficiency with or without solid-liquid separation after alkali treatment was checked by use of the residue (47% of cellulose and 53% of lignin, etc.) obtained by the dilute sulfuric acid treatment.

Every 10 g per dry weight of residual substances, which carried out dilute-sulfuric-acid processing, were put into seven 200 mL culture flasks, and 3% sodium hydroxide solution was mixed so that solid concentration might become 10%. After agitating at room temperature for 3 hours, hydrogen peroxide was added in the range of 20-315 mg-H₂O₂/g-dry matter, and agitating was continued for 12 hours (process A).

Every 10 g per dry weight of residual substances, which carried out dilute-sulfuric-acid processing, were put into five 200 mL culture flasks, and 3% sodium hydroxide solution was mixed so that solid concentration might become 10%. It separated into the solid and the liquid, after agitating at room temperature for 3 hours. The collected solid was moved to another culture flask, and it mixed in 3% sodium hydroxide solution. Then, hydrogen peroxide was added in the range of 50-150 mg-H₂O₂/g-dry matter, and agitating was performed for 12 hours.

(Process B)

The solid collected from each culture flask was mixed to the acetic acid buffer solution of pH 4.8, and temperature was set as 40° C. Cellulase (GC220 manufactured by Genencor, Inc) was added so that it might become the activity of 15 FPU per 1 g of solids, and agitating was continued for 144 hours. The glucose concentrations in a liquid were measured by GPC. The saccharification yields from cellulose were calculated.

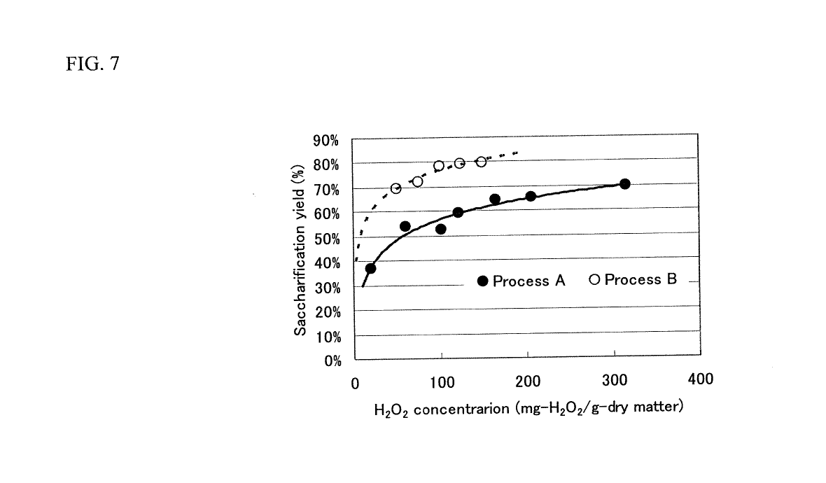

The experimental results are shown in FIG. 7. In the process B which added hydrogen peroxide to the solid content separated after alkali treatment, the result with high saccharification yield was obtained compares with Process A.

The present invention makes it possible to provide an inexpensive and efficient method for saccharification of woody biomass. According to the saccharification method of the invention, the saccharification yields of hemicellulose and cellulose contained in woody biomass can be greatly improved and energy can be saved because the reaction of saccharification is performed under normal temperature/pressure conditions. Furthermore, in each step of the saccharification method of the invention, since the concentration of an alkali compound or an oxidant is low, the equipment can be simplified and its operational safety is high. Moreover, the present invention provides a method of producing ethanol. In the ethanol production method of the present invention, woody biomass can be efficiently converted into energy. Therefore, it is effective to recycle a natural resource.

What is claimed is:

1. A method for saccharification of woody biomass comprising hydrolysis treatment and an enzymatic treatment, which comprises a pretreatment process comprising the steps of:
hydrolyzing woody biomass to separate the resultant reaction product into a primary sugar solution and a residue, and
mixing the residue with an aqueous alkaline solution, and then with an oxidant capable of generating active oxygen.

2. A method for saccharification of woody biomass comprising hydrolysis treatment and an enzymatic treatment, which comprises a pretreatment process comprising the steps of:
hydrolyzing woody biomass to separate the resultant reaction product into a primary sugar solution and a residue,
separating the resultant reaction product into a primary sugar solution and a residue,
mixing the residue with an aqueous alkaline solution to obtain a mixture,
separating the mixture into a secondary residue and a solution, and
mixing an oxidant capable of generating active oxygen with the obtained secondary residue in maintaining alkaline state.

3. The method for saccharification of woody biomass according to claim 1 or 2, further comprising a step of subjecting a solid component of the mixture obtained in the pretreatment process to an enzymatic treatment with cellulase.

4. The method for saccharification of woody biomass according to claim 1 or 2, wherein time interval after mixing the residue with the aqueous alkaline solution until mixing the oxidant capable of generating active oxygen is set at 1 to 30 hours.

5. The method for saccharification of woody biomass according to claim 1 or 2, wherein the hydrolysis treatment is performed with dilute sulfuric acid.

6. The method for saccharification of woody biomass according to claim 1 or 2, wherein, in the pretreatment process, the residue is mixed with an aqueous alkaline solution at pH 10 to 13.

7. The method for saccharification of woody biomass according to claim 1 or 2, wherein the oxidant capable of generating active oxygen is hydrogen peroxide.

8. The method for saccharification of woody biomass according to claim 7, wherein 0.1% or more hydrogen peroxide is used as the hydrogen peroxide.

9. The method for saccharification of woody biomass according to claim 3, wherein the solid component is subjected to enzymatic treatment with cellulase to obtain a secondary sugar solution.

10. The method for saccharification of woody biomass according to claim 1 or 2, wherein the solid component of the mixture obtained in the pretreatment process is subjected to enzymatic treatment with cellulase and ethanol fermentation treatment to obtain ethanol.
